(19) United States
(12) Patent Application Publication
(10) Pub. No.: US 2021/0187136 A1
(43) Pub. Date: Jun. 24, 2021

(54) Splint Bath Assembly

(71) Applicants: Debbie Joles, Hixson, TN (US); Chad Morgan, Chattanooga, TN (US)

(72) Inventors: Debbie Joles, Hixson, TN (US); Chad Morgan, Chattanooga, TN (US)

(21) Appl. No.: 17/099,658

(22) Filed: Nov. 16, 2020

Related U.S. Application Data
(60) Provisional application No. 62/935,947, filed on Nov. 15, 2019.

(51) Int. Cl. A61L 2/00; A61F 5/058

(52) U.S. Cl. CPC A61L 2/0088; A61L 2/0047; A61L 2202/21; A61L 2202/122; A61F 5/058

(57) ABSTRACT

Disclosed herein is a novel splint bath assembly for creating a sanitary and effective sterilization conditions for preparing material for splints and casts. In one embodiment, the splint bath assembly includes a reservoir, an ultraviolet light system, circulation pump, and a heat exchanger. The circulation pump creates convection flow to continuously circulate water in the reservoir over the ultraviolet light system to sanitize the water in the reservoir. Additionally, the splint bath assembly can include an heat sanitation mode that generally heats the water in the reservoir to a relatively high temperature for several minutes to offer an additional method for reducing or eliminating organisms found in this environment. The splint bath assembly can be arranged to automatically and efficiently drain water from the splint bath to facilitate clinician clean up.

### CROSS-REFERENCE TO RELATED APPLICATION

This application claims priority to Provisional Patent Application No. 62/935,947, filed on Nov. 15, 2019, and titled “Splint Bath Assembly,” the disclosure of which is incorporated herein by reference in its entirety.

### TECHNICAL FIELD

The present disclosure generally relates to novel splint bath assemblies. More specifically, the present disclosure relates to novel splint bath assemblies that provide a sanitary and effective sterilization environment to facilitate repeatable processes for use in a clinical setting.

### BACKGROUND

A splint bath, also commonly referred to as a splint pan, is a device that assists medical professionals in many fields such as occupational theory, orthopedic surgery, physical theory, emergency medicine, sports medicine, and oncology. Splint bath assemblies are commonly used in hospitals, clinics and other medical facilities to prepare materials for splints and casts or other uses that immobilize a section of a patient's body. Splint baths are often used to heat and soften sheets of thin plastic material (generally 1/16 inch to 3/16 inch in thickness) until the sheets are sufficiently pliable to conform to the contours of a patient's body. Once the plastic material is sufficiently pliable, the plastic sheets can be applied to a patient's body, where the plastic sheets harden after a short period of time to stabilize a splint or form a cast or otherwise immobilize a section of the patient's body.

FIG. 1 schematically illustrates such heated and softened sheets of plastic being applied to stabilize a splint on the wrist and hand of a patient. FIG. 2 schematically illustrates a heated and softened plastic matrix used to immobilize a patient's head prior to undergoing an oncology procedure. FIGS. 1 and 2 depict a pair of applications for plastic materials prepared in a splint bath; however, it will be appreciated that these are but two such examples and that there are many more practical uses for such prepared plastic materials.

A splint bath typically includes a reservoir for holding water and a heating element to heat the water. Once the water in the reservoir is heated, plastic sheets can be submerged in the water in the reservoir for heating and softening of the plastic material. During certain medical procedures softened plastic sheets are removed from the splint bath assembly and applied directly to sections of the human body, where the softened plastic sheets adhere to the contours of the body. However, as part of the medical procedure, it is not uncommon that once a plastic sheet is initially applied to the human body, it may not sufficiently adhere to the contours of the body. One common cause is that current splint bath assemblies do not uniformly heat the water in the reservoir, resulting in inconsistent in plastic sheeting removed from such current splint bath assemblies. In such a situation, the plastic sheet is removed from the body and again immersed in the splint bath assembly to further soften the plastic for better positioning on the section of the human body.

For current splint baths, the water in the reservoir is typically changed infrequently and is used for attending to multiple patients. This is due to the design and structure of current split baths, neither of which facilitate an efficient process for changing the water within the splint bath. Additionally, current splint baths do not effectively regulate temperature throughout the water in the reservoir nor sufficiently circulate the water in the reservoir. As will be appreciated, such a scenario can lead to unintended health issues. For example, over time as multiple patients are treated using a splint bath assembly, viruses, pathogens, bacteria, microbes, and other harmful organisms can grow in the stagnant and unevenly heated water in the reservoir. In such a scenario, such microorganisms can adhere to the plastic sheets being prepared in the splint bath and be transferred from the splint bath directly to the patient during treatment. It will be understood that such cross-contamination can cause the patient to develop infections, viral illnesses, or other infirmities by the introduction of unwanted microorganisms to the patient's body.

There exists a need in the field of splint bath design and methods for manufacturing and using splint bath assemblies for novel designs and methods that prevent and/or mitigate the growth of organisms in the reservoir of the splint bath assembly and to evenly and consistently heat the water in the reservoir while it is in use for treating patients. The novel splint bath assemblies and methods of use disclosed herein are directed to assemblies that include features and functionality that meet these long sought after needs.

### SUMMARY

Disclosed herein is a novel splint bath assembly for creating a sanitary and effective sterilization conditions for preparing material for splints and casts. In one embodiment, the splint bath assembly includes a reservoir, an ultraviolet light system, circulation pump, and a heat exchanger. The circulation pump creates convection flow to continuously circulate water in the reservoir over the ultraviolet light system to sanitize the water in the reservoir. Additionally, the splint bath assembly can include an heat sanitation mode that generally heats the water in the reservoir to a relatively high temperature for several minutes to offer an additional method for reducing or eliminating organisms found in this environment. The splint bath assembly can be arranged to automatically and efficiently drain water from the splint bath to facilitate clinician clean up.

### BRIEF DESCRIPTION OF THE DRAWINGS

FIG. 1 schematically illustrates a medical professional applying pliable sheets of plastic to support a splint on the wrist and hand of a patient.

FIG. 2 schematically illustrates the use of a plastic matrix to immobilize a patient's head prior to undergoing an oncology procedure.

FIG. 3 schematically illustrates a perspective view of an exemplary embodiment of a splint bath assembly in accordance with the present disclosure.

FIG. 4 schematically illustrates another perspective view of the splint bath assembly of FIG. 3.

FIG. 5 schematically illustrates a top view of the splint bath assembly of FIG. 3.

FIG. 6 schematically illustrates a perspective view of inner components of the splint bath assembly of FIG. 3.

FIG. 7 schematically illustrates another perspective view of inner components of the splint bath assembly of FIG. 3.

FIG. 8 schematically illustrates an exemplary ultraviolet lighting system for use with the splint bath assembly of FIG. 3.

FIG. 9 schematically illustrates a rear view of the splint bath assembly of FIG. 3.

FIG. 10 schematically illustrates additional perspective views of the splint bath assembly of FIG. 3.

FIG. 11 schematically illustrates plan view of the splint bath assembly of FIG. 3.

FIG. 12 is a graph depicting the relationship between bacteriophage MS2 and time.

FIG. 13 is a graph depicting the relationship between mycobacterium terrae and time.

FIG. 14 schematically illustrates an exemplary embodiment of a display of a control panel for use with the splint bath assembly of FIG. 3.

FIG. 15 schematically illustrates another view of the display of the control panel of FIG. 14.

FIG. 16 schematically illustrates another view of the display of the control panel of FIG. 14.

FIG. 17 schematically illustrates another view of the display of the control panel of FIG. 14.

### DETAILED DESCRIPTION

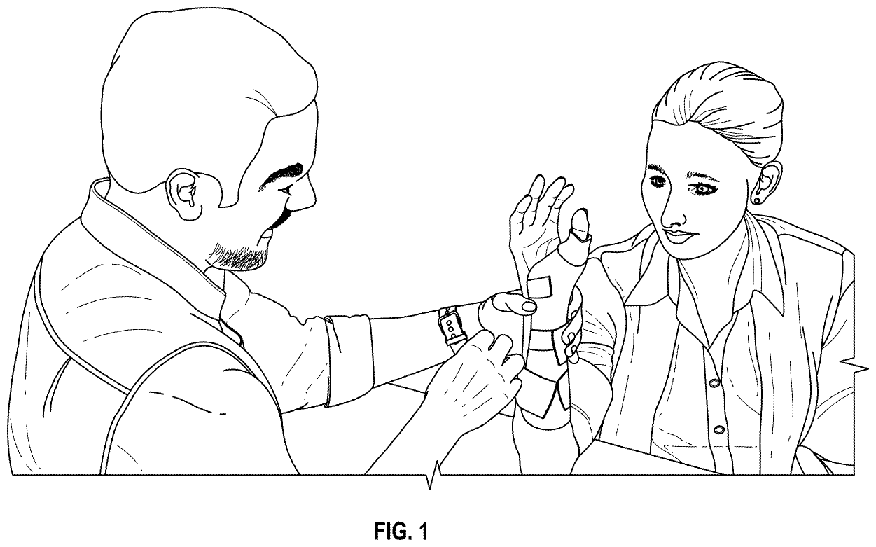

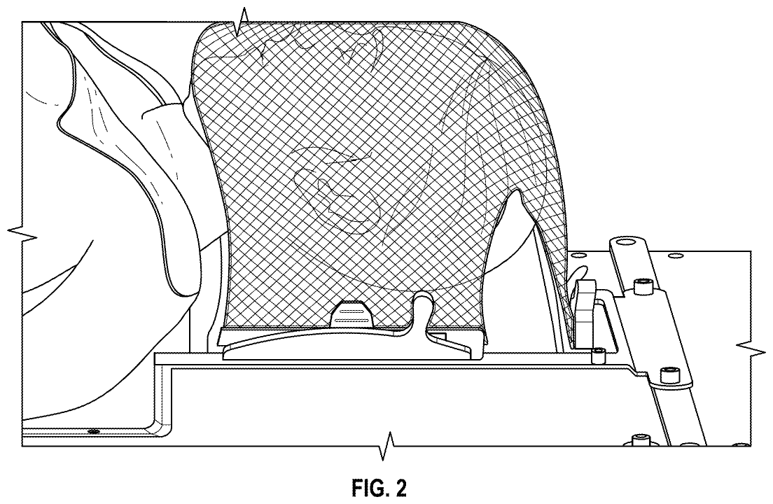

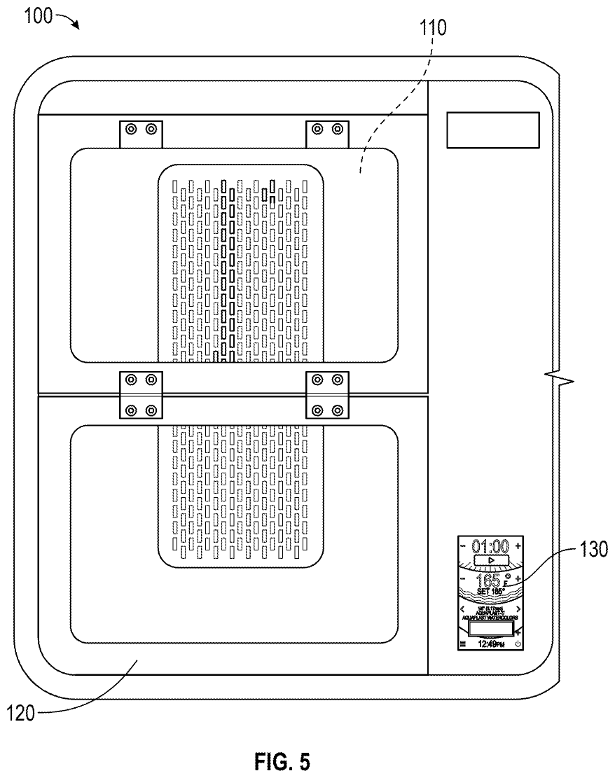

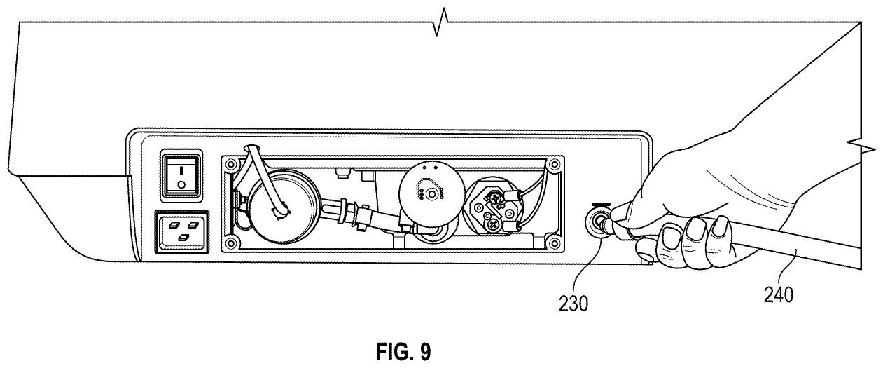

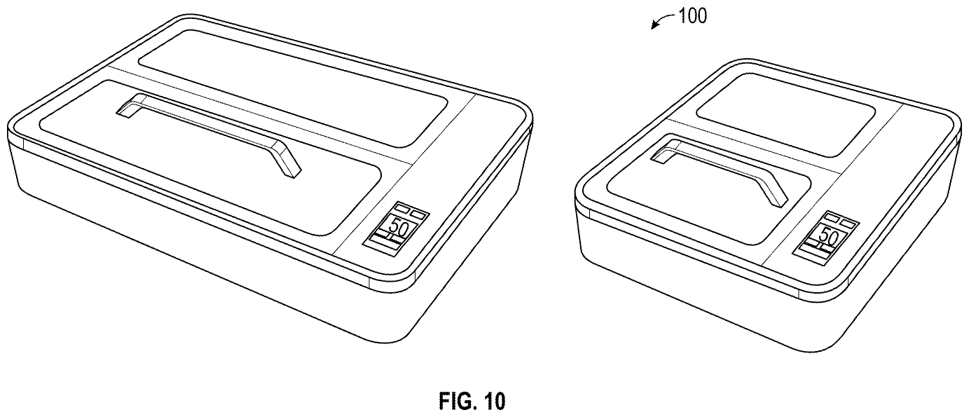

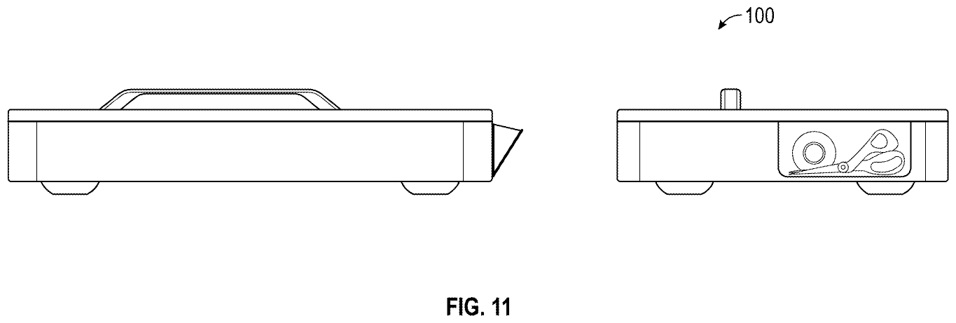

The apparatus, systems, arrangements, and methods disclosed in this document are described in detail by way of examples and with reference to the figures. It will be appreciated that modifications to disclosed and described examples, arrangements, configurations, components, elements, apparatus, methods, materials, etc. can be made and may be desired for a specific application. In this disclosure, any identification of specific techniques, arrangements, method, etc. are either related to a specific example presented or are merely a general description of such a technique, arrangement, method, etc. Identifications of specific details or examples are not intended to be and should not be construed as mandatory or limiting unless specifically designated as such. Selected examples of novel splint bath assemblies are hereinafter disclosed and described in detail with reference made to FIGS. 1-17.

A novel splint bath assembly disclosed herein is designed to maximize the sanitary and effective sterilization conditions of the splint bath assembly to provide a high degree of temperature control and infection control for medical professionals and their patients. This is accomplished by providing certain functionality to create a constant desired temperature across the water in the splint bath and redundant systems that eliminate or reduce the presence of organisms within the splint bath assembly. For example, the splint bath assembly includes an ultraviolet light system, circulation pump, and heat exchanger. The circulation pump creates convection flow to continuously circulate the water in the reservoir over the ultraviolet light system to sanitize the water in the reservoir. As will be subsequently discussed, a study of the novel splint bath assembly has concluded that such a process eliminates up to 99.99% microorganisms in the water circulating through the reservoir. Secondarily, the splint bath assembly can include an heat sanitation mode that generally heats the water in the reservoir to a relatively high temperature (approximately 185 degrees Fahrenheit) for several minutes to offer an additional method for reducing or eliminating organisms found in this environment. Finally, the splint bath assembly is arranged to automatically and efficiently drain water from the splint bath to facilitate clinician clean up.

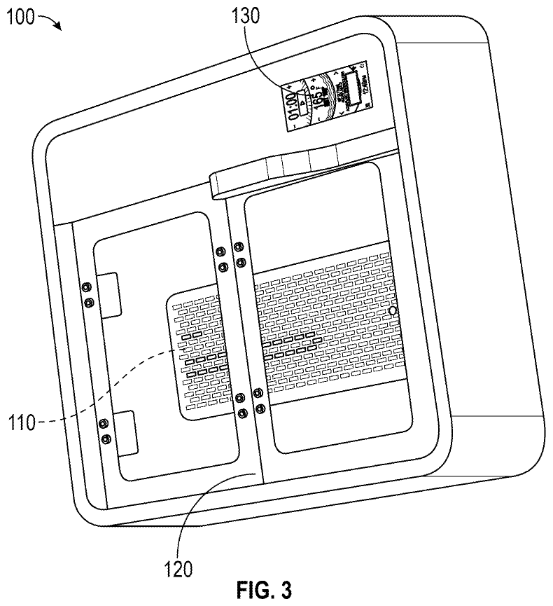

FIGS. 3-11 illustrated an exemplary embodiment of a splint bath assembly 100. In one example, the splint bath assembly 100 is generally fabricated from a thermoset composite material that provides superior durability, is generally non-porous so as to resist the penetration of microbes, has a generally pleasing aesthetic appearance, and is approved by the United States Food and Drug Administration for use as a material for medical devices. The splint bath assembly 100 includes a reservoir 110 for holding water, a lid 120 for covering and uncovering the reservoir 110, and a control panel 130 for controlling the functionality of the splint bath assembly 100 such as establishing or adjusting settings and initiating various functions and procedures. Such adjustable settings include temperature and time settings, and such functions and procedures include evacuating and refilling water from the reservoir, ultraviolet light cycling of the water in the reservoir 110, and cleaning cycles for the splint bath assembly 100. The reservoir is typically sized to accommodate between five and ten gallons of water, but can be arranged to other sizes depending on the specific purpose of the splint bath assembly.

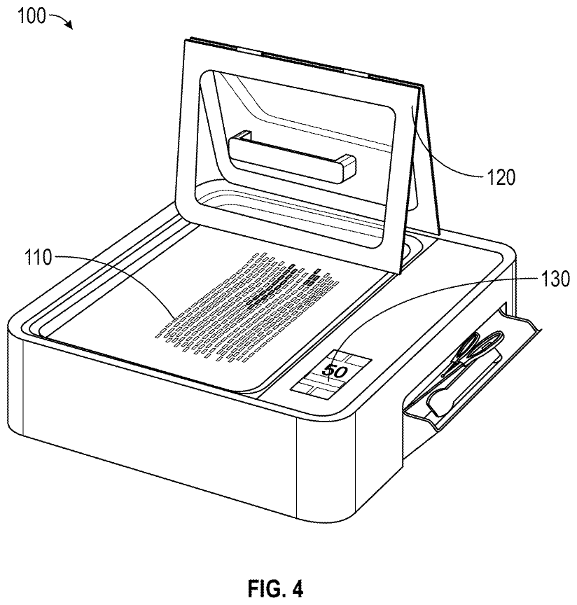

In addition to the durability provided by the thermoset composite material, the material also provides a flammability resistant property for enhanced safety and an insulating property to assist in maintaining the desired temperature of the water in the reservoir 110. The insulating property provides for minimal heat dissipation from the water in the reservoir 110 thus, providing better control of the temperature of the water bath. As illustrated by comparing FIG. 3 to FIG. 4, the lid 120 is hinged such that it can be easily moved between a closed (FIG. 3) and an open (FIG. 4) position. The lid 120 is generally transparent to provide visibility into the reservoir 110. Such visibility can be important for the user to view plastic sheets being treated in the splint bath assembly 100. In one example, the lid 120 is fabricated from tempered glass. As will be appreciated, when the lid 120 is closed, it serves to assist in maintaining the desired temperature of the water in the reservoir 110, and when the lid 120 is open, it provides easy access to the reservoir 110 to place plastic sheets into the reservoir 110, inspect plastic sheets in the reservoir 110, and remove such plastic sheets from the reservoir 110 when treatment is completed.

The temperature of the water in the reservoir 110 is set and maintained through the use of an internal temperature control system that provides for the temperature of the water to be monitored in real-time with tight tolerances to insure the desired temperature is maintained. Such real-time monitoring can be done by a recessed thermostat located such that it is submerged in the water bath. The splint bath assembly 100 includes a heating coil 140 positioned within the reservoir 110 and submerged in the water bath that functions as a heat exchanger that heats the water bath through convection technology. The heat system can include a user interface in communication with an internal probe located at or near the heating coil that provides for tight temperature control to a tolerance of +2 degrees Fahrenheit. Additionally, the user interface includes functionality for inputting manufacturer protocols to control the temperature of the splint bath assembly 100. As will be further discussed, the splint bath assembly 100 includes a system that circulates the water in the reservoir 110 to assist the convection heating process by forcing water to continuously flow into contact with the heating coil 140.

The splint bath assembly 100 is arranged to have a sanitation mode that can be initiated automatically or manually. When the sanitation mode is initiated, the temperature of the water in the reservoir 110 is increased in one example to approximately 185 degrees Fahrenheit to kill any microorganisms in the water. With the circulation of the water within the reservoir 110, such high temperatures are achieved evenly throughout the water bath for effective microorganism management. In other embodiments, the temperature range can be raised above 185 degrees Fahrenheit based on testing and/or the experience and judgment of the medical professional using the splint bath assembly 100.

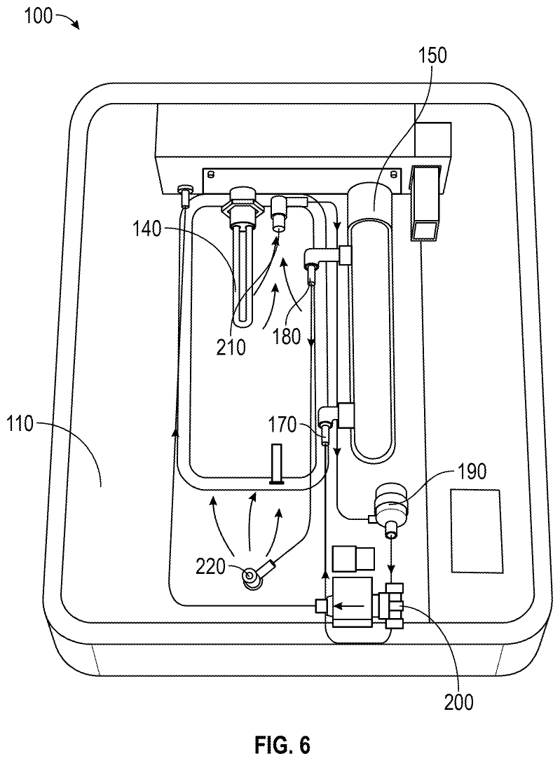

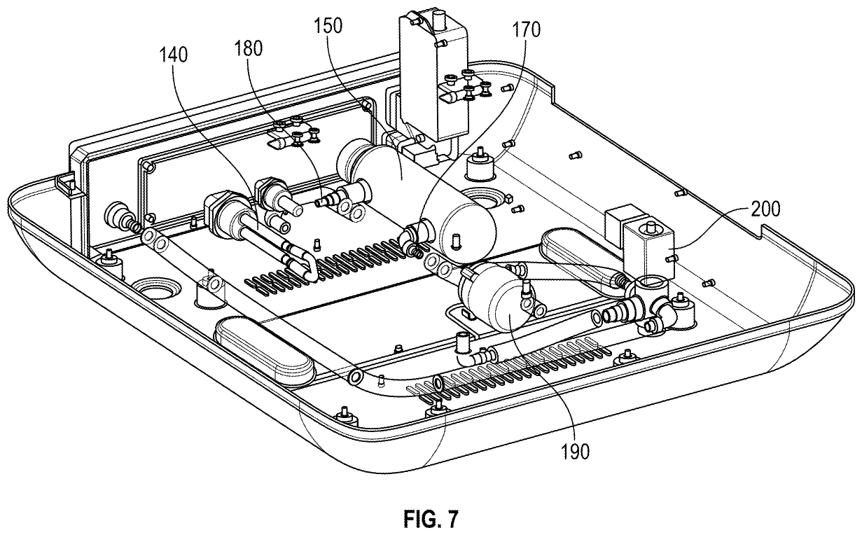

In addition to the sanitation mode facilitated by elevated temperatures, the splint bath assembly 100 includes an UV-C light effective sterilization system 150. This system 150 includes an ultraviolet light source 160 (illustrated in FIGS. 6-8). The ultraviolet light source 160 emits light with an approximately 254 nanometer wavelength. Such light kills approximately 99.99 percent of microbes inoculated by the light. The UV-C light effective sterilization system 150 is arranged to bring water into close proximity to the ultraviolet light source 160. To this end, the UV-C light effective sterilization system 150 includes an inlet port 170 and an outlet port 180. A pump 190 and valve 200 combination (illustrated in FIGS. 6 and 7) circulate water through the UV-C light effective sterilization system 150 and past the ultraviolet light source 160, where the ultraviolet light source 160 sterilizes the passing water. Alternatively, an added inlet port 210 and outlet port 220 can be positioned at opposite ends of the reservoir 110 to facilitate the general circulation of water throughout the reservoir 110. The flow rate of the pump 190 can be set such that the total volume of water in the reservoir 110 circulates through the UV-C light effective sterilization system 150 approximately every two minutes. It will be appreciated that such rapid circulation will maintain a sanitary and effective sterilization environment within the reservoir 110 and will provide infection control when the splint bath assembly 100 is used to heat and soften plastic sheets used for splints, casts, and to immobilize patients. In other examples, the flow rate of the pump 190 can be set to accomplish appropriate infection control for the specific use of the splint bath assembly 100.

Testing of the novel splint bath assembly 100 disclosed herein demonstrated that the splint bath 100 assembly achieves an effective sterilization rate. The splint bath assembly 100 was tested for effectiveness against several different microorganisms, including Staphylococcus epidermidis (“S. epidermidis”), Escherichia coli (“E. coli”), bacteriophage MS2 (“MS2”), and mycobacterium terrae (“M. terrae”). The microorganism was tested under two protocols—a maintenance cycle and a sanitization cycle, further detailed below in Table 1.

TABLE 1

| Parameter | Maintenance Cycle | Sanitization Cycle |
|---|---|---|
| Temperature | 140 ± 1° F. | 185 ± 1° F. |
| Time intervals | 0 minutes | 0 minutes |
| | 1 minute | 1 minute |
| | 10 minutes | 10 minutes |
| | 30 minutes | |
| | 60 minutes | |
| Application of UV light | Yes | Yes |

S. epidermidis and E. coli were tested in the maintenance cycle, and MS2 and M. terrae were tested in the sanitization cycle. The results of the testing are illustrated in the table and accompanying figures.

As is illustrated in the Table 2 below, when the maintenance cycle is applied, S. epidermidis and E. coli are effectively managed. An initial concentration of S. epidermidis of 76,666 colony-forming units per milliliter (“CFU/ml”) was reduced to 163 CFU/ml in one minute and to zero in ten minutes, and an initial concentration of E. coli of 1,033,333 CFU/ml was reduced to 895 CFU/ml in one minute and to zero in ten minutes.

TABLE 2

| | | | Test Microorganism | |
|---|---|---|---|---|
| | | | S. epidermidis | E. coli |
| Test Device | Contact Time | Cycle | ATCC 12228 | ATCC 11229 |
| UV Enabled | Time Zero | Maintenance | 76,666 | 1,033,333 |
| Splint Bath | 1 Minute | Cycle | 163 | 895 |
| Assembly | 10 Minutes | | 0 | 0 |
| | 30 Minutes | | 0 | 0 |
| | 60 Minutes | | 0 | 0 |

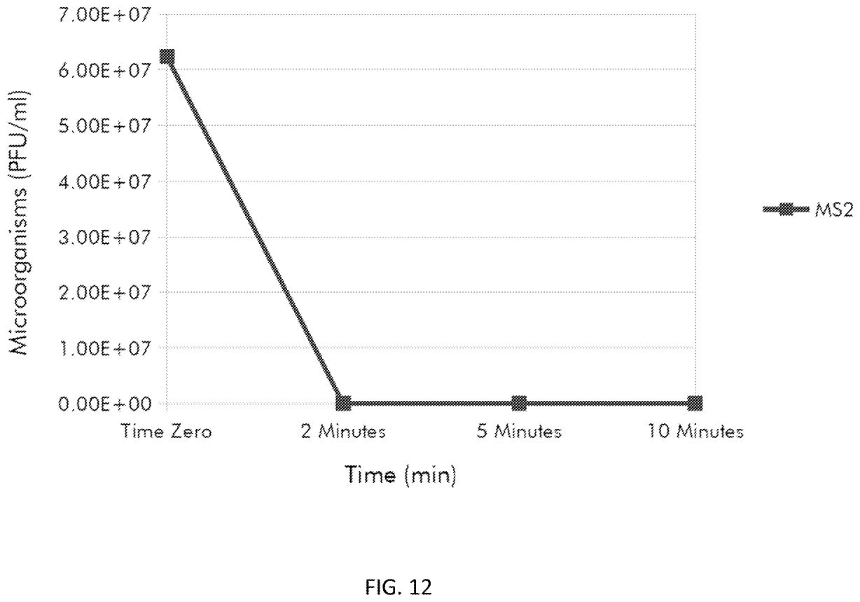

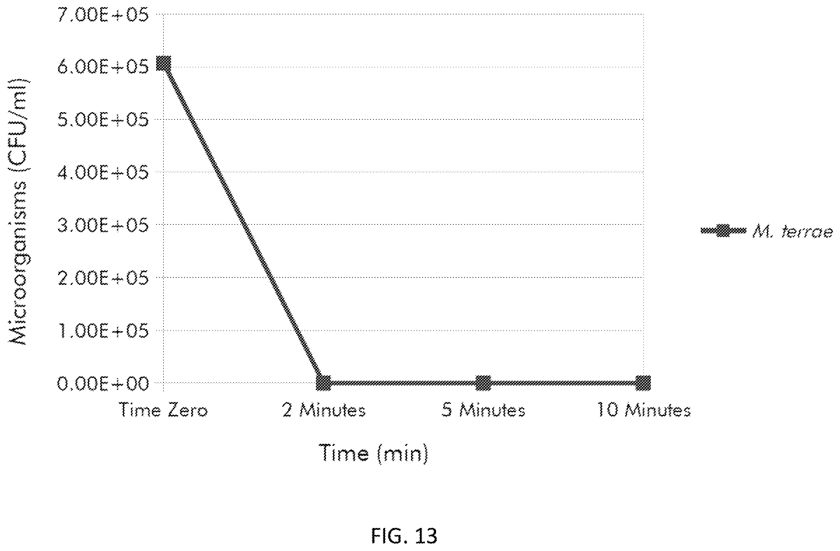

As is illustrated in graphs shown in FIGS. 12 and 13, when the sanitization cycle is applied, all tested microorganisms are effectively managed. As illustrated in the graph shown in FIG. 12, an initial concentration of MS2 of 62,333,333 CFU/ml was reduced to zero in two minutes. As illustrated in the graph shown in FIG. 13, an initial concentration of M. terrae of 606,666 was reduced to zero in two minutes.

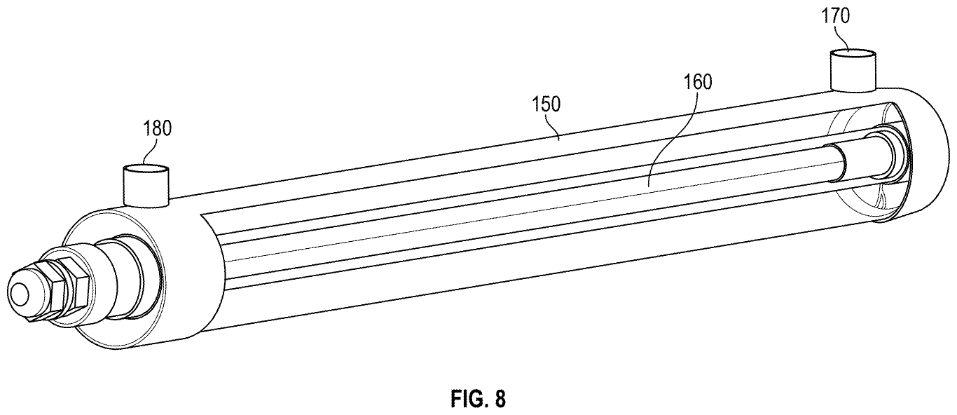

As previously noted, the splint bath assembly 100 also includes the functionality of total evacuation of all water from the reservoir 110 and the refilling of the reservoir 110 with fresh water. The splint bath assembly 100 uses the pump 190 and valve combination 200 (illustrated in FIG. 6) to quickly and controllably evacuate the water from the reservoir 110 and refill the reservoir 110. An outlet port 230 (illustrated in FIG. 8) can be integrated into the body of the splint bath assembly 100 and connected to tubing 240, which in turn can be connected to a drain and/or a water source.

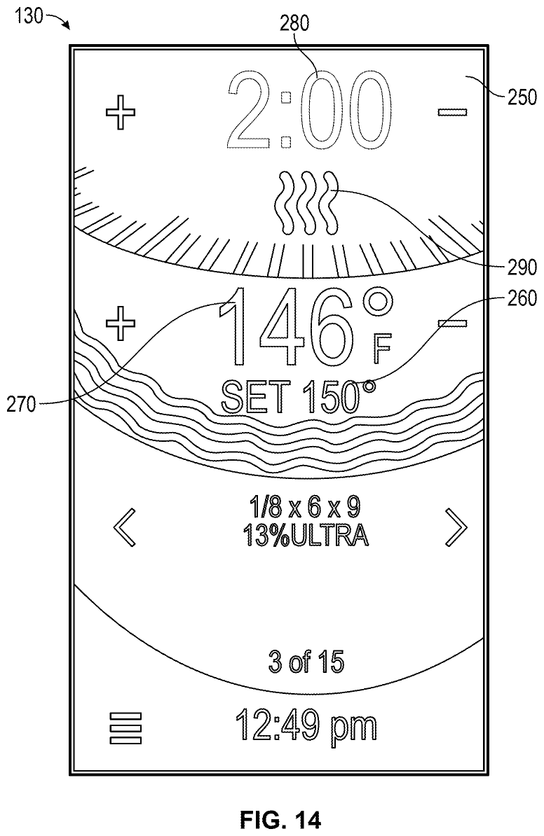

As previously noted, the split bath assembly 100 includes a control panel 130. The control panel 130 includes a display screen 250, which in one example is a light emitting diode (LED) touch screen user interface (illustrated in FIGS. 14-17). The display screen can be used for a variety of functions such as to adjust various parameters such as temperature of the water in the reservoir 110, timing for sanitation mode, and flow rate of water through the pump located in the reservoir 110. The display screen 250 can further record preset parameters either programmed prior to the time of sale or subsequently entered by a medical professional or other trained user. The display screen 250 can also include a button for initiating the auto drain and auto fill function. The functionality of the display screen 250 will be discussed in greater detail below. However, FIGS. 14-17 will be cited to generally describe the functionality of the display screen 250 and control panel 130.

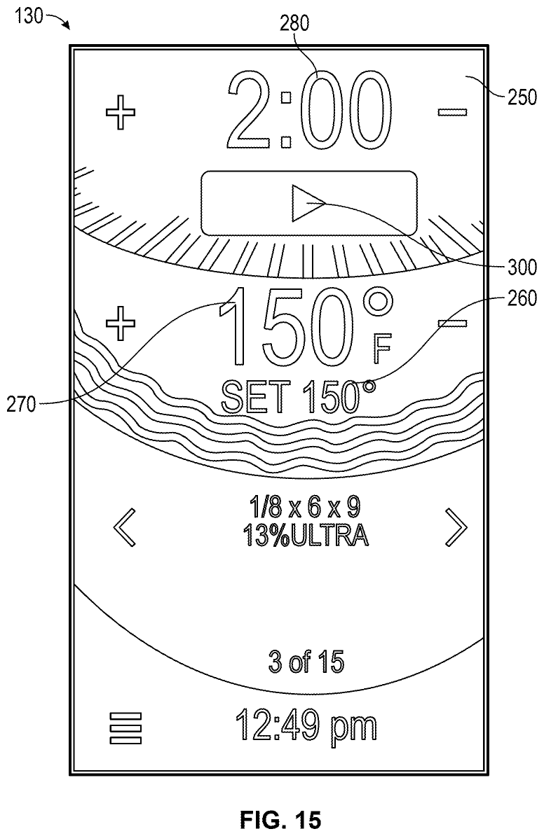

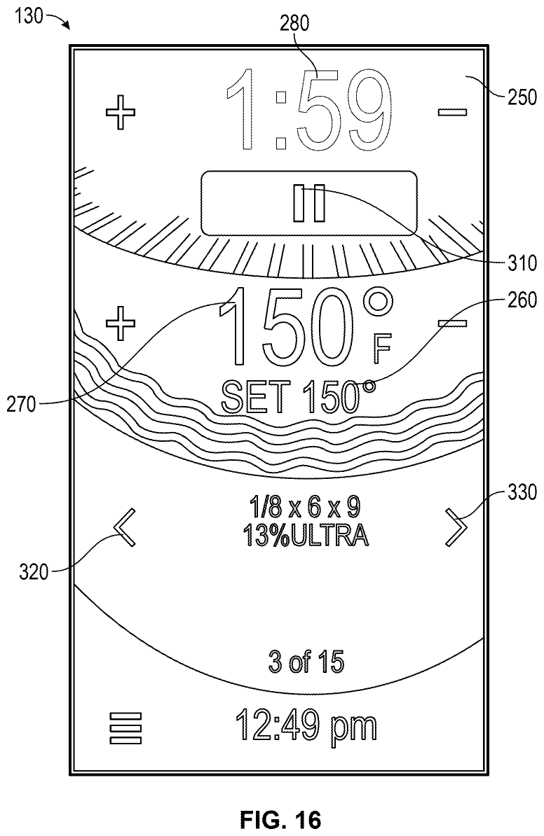

FIG. 14 illustrates one example of an arrangement of the display screen 250. The display screen 250 displays a set point 260 representing the desired temperature of the water bath, the actual temperature 270 of the water bath, and the desired duration 280 for the plastic sheets to remain in the water bath. The display screen 250 includes an icon 280 (wavy lines) to indicate the water bath is still heating. As illustrated in FIG. 15, once the unit reaches its desired temperature, the process is ready to be initiated. A medical professional or other trained user can initiate the process by touching a green start arrow 300 that is displayed on the display screen 250 when the desired temperature 260 is reached. As illustrated in FIG. 16, the process can be paused by touching a blue pause button 310 that is displayed on the display screen 250. Additionally, there are left 320 and right 330 arrow icons that allow the user to cycle through the preset options. Generally, the control panel 130 can include functionality that displays: target temperature of water bath; actual temperature of water bath; selection between Fahrenheit and Celsius temperature scales, time buttons, timer start/stop button, audio cues, language selection, timer settings, and temperature adjustment.

In one example, the display screen 250 of the control panel 130 lights up when the splint bath assembly 100 is turned on. The functionality of the control panel 130 includes providing users with options to customize the splint bath assembly 100 according to their needs. For example, the home screen includes information such as session time, current water temperature, set temperature, presets, current time, and a menu icon, but can be reconfigured to the user's desired arrangement. The splint bath assembly 100 can include a number of predetermined sets of parameters (temperature and time) that match recommendations from the manufacturers of various plastic sheets (to be used for casts, splits, etc.). Such presets are for the convenience of the user and should yield the best results when using the splint bath assembly to heat and soften such plastic sheets. However, the splint bath assembly has the flexibility for the user to modify such presets based on the user's own experience and professional medical opinion.

The display screen can include a number of functional icons. For example, plus and minus icons (+ and −) that can be conveniently depressed for the user to make changes to settings and parameters and left and right arrow icons (< and >) to toggle through settings. As noted earlier, the display screen 250 can include wavy vertical lines to indicate that the water bath is heating but not yet reached its desired temperature, a green box with an arrow to indicate desired temperature has been reached, and a pause button that stops the process as desired by the user.

The menu can include a number of features, such as functionality to set auto on/off, which allows the user to set an automatic turn on time and turn off time for specific days of the week. Such functionality is useful to preheat the water bath to reach set temperature prior to a session starting. The menu can include a water evacuation start button, which when depressed activates the pump to evacuate the water via the drain hose. The pump will stop when the reservoir is empty; however, drainage can be stopped at any time by the user depressing a “stop” button.

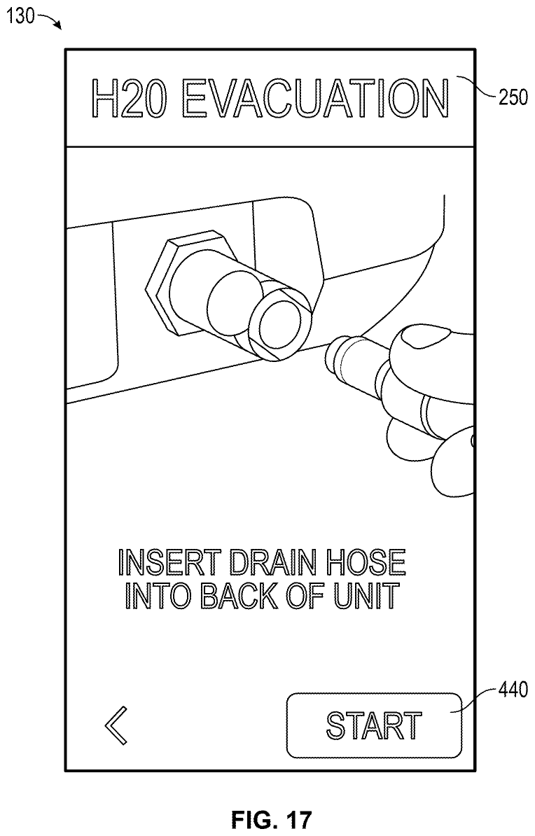

FIG. 17 illustrates another example of an arrangement of the display screen 250. The display screen 250 can be used to initiate various operations of the splint bath assembly and provide instructions to accompany such operations. For example, the display screen 250 can be used to initiate the operation of evacuating all water from the reservoir of the splint bath assembly. As illustrated, the user is instructed to insert the drain hose into the back of the splint bath assembly prior to initiated the operation. Such instructions can be important to effective operation of the splint bath assembly. The evacuation operation can be initiated by pressing on the green “start” button 400.

For the sanitation cycle, it is best practice to be run daily. It may be convenient to set the sanitation cycle to run each evening during off hours so that the splint bath assembly is prepared for operation at the start of the next work day. The times and days to run the sanitation cycle can be set by the user. As noted above, when the sanitation cycle is engaged, the splint bath assembly will heat the water to a high temperature (approximately 200 degrees Fahrenheit) to kill microbes in the water bath. The water temperature is displayed on the control panel during the sanitation cycle to alert the user that the splint bath assembly is operating at high temperature. During the sanitation cycle, the pump and ultraviolet lighting system are disengaged. After the sanitation cycle is completed, the splint bath assembly can automatically turn off or be cooled to last preset used (depending upon user preferences). The sanitation cycle can also be manually initiated at any time by the user.

The foregoing description of examples has been presented for purposes of illustration and description. It is not intended to be exhaustive or limiting to the forms described. Numerous modifications are possible in light of the above teachings. Some of those modifications have been discussed, and others will be understood by those skilled in the art. The examples were chosen and described in order to best illustrate principles of various examples as are suited to particular uses contemplated. The scope is, of course, not limited to the examples set forth herein, but can be employed in any number of applications and equivalent devices by those of ordinary skill in the art.

We claim:

1. A splint bath assembly as described and shown herein.

2. A method of using a splint bath assembly as described and shown herein.
